(19) United States
(12) Patent Application Publication
(10) Pub. No.: US 2004/0248990 A1
(43) Pub. Date: Dec. 9, 2004

(54) Ketone peroxide compositions

(76) Inventors: Bernhard De Vries, Hulshorst (NL); Andreas Petrus Van Swieten, Velp (NL); Ejaz Ahmed Syed, Deventer (NL)

Correspondence Address: OLIFF & BERRIDGE, PLC, P.O. BOX 19928, ALEXANDRIA, VA, 22320, US

(21) Appl. No.: 10/501,147

(22) PCT Filed: Feb. 5, 2003
(86) PCT No.: PCT/EP03/01121

(30) Foreign Application Priority Data
Feb. 22, 2002 (EP) 02075731.6

(51) Int. Cl. A61K 31/12

(52) U.S. Cl. 514/675

(57) ABSTRACT

The invention pertains to a composition of a ketone peroxide comprising a) a peroxide derivative of the formula HOO—C(R₁)(R₂)—OOH wherein R₁ is a branched or unbranched alkyl group with 1 to 4 carbon atoms or alkenyl group with 2 to 4 carbon atoms; and R₂ is a branched or unbranched alkyl or alkenyl group with 5 to 12 carbon atoms; and b) a branched or unbranched hydrocarbon solvent; the peroxide derivative of a) having a solubility more than 40 g in 100 g of the solvent of b) at 20° C.; and comprises less than 10 wt % of a peroxide derivative of the formula HOO—C(R₁)(R₂)—OO—C(R₁)(R₂)—OOH, wherein R₁ and R₂have the previously given meanings.

The present invention pertains to a composition of a ketone peroxide of the formula HOO—C(R₁)(R₂)—OOH, to the corresponding bis- and mono-peroxyesters, bis- and mono-peroxycarbonate's, and mixed peroxyester-peroxycarbonates derived from said ketone peroxides, and to a method for the preparation thereof, to the use of said composition.

Ketone peroxides of formula HOO—C(R₁)(R₂)—OOH, the so-called Type 4 ketone peroxides (such as 1,4-dimethylpentylidene dihydroperoxide), are in view of their low production costs highly interesting starting materials for various peroxides such as bis-peroxyesters, bis-peroxy carbonates or mixed peroxyester peroxycarbonate derivatives. Such Type 4 ketone peroxide derivatives are for instance disclosed in WO 9932442, wherein R₁ and R₂ are independently selected from hydrogen, C₁-C₂₀ alkyl, C₃-C₂₀ cycloalkyl, C₆-C₂₀ aryl, C₇-C₂₀ aralkyl, and C₇-C₂₀ alkaryl, of which the methylisobutyl and methylisopropyl ketone peroxides were the only ones that were specifically disclosed. In U.S. Pat. No. 3,326,809 similarly methyl ethyl, diisopropyl, methyl amyl, and methylhexyl ketone peroxides were disclosed, whereas in U.S. Pat. No. 3,151,170 the ethyl isoamyl ketone peroxide was disclosed. Since these ketone peroxides comprise two hydroperoxy groups of significantly different acidity, the two hydroperoxy groups can highly selectively be reacted separately with reactive electrophilic compounds such as acid chlorides or alkyl chloroformates. This high selectivity is not observed in the case that di-hydroperoxy alkyl peroxides are used as starting material, such as the commercially widely used 2,5-dihydroperoxy-2,5-dimethylhexane. The small difference between the acidity of the hydroperoxy groups of this alkanediol-derived molecule prevents high selectivity upon reaction with electrophiles. Using ketone peroxides of formula HOO—C(R₁)(R₂)—OOH, multi-half-life peroxides (i.e. peroxides with different reactivities in the same molecule) are so within reach easily. These peroxides are of high interest in the polymerization industry, especially in applications wherein broad temperature ranges are applied during manufacturing such as in low-density polyethylene polymerization (LDPE), and in the production of PVC, polymethacrylate, polystyrene, polyethylene, copolymers, and thermoset polymers.

It is known that ketone peroxides of the formula HOO—C(R₁)(R₂)—OOH are highly soluble in polar solvents but only slightly soluble or insoluble in apolar hydrocarbon solvents. Thus, Type 4 ketone peroxides derived from commercially readily available ketones such as methylethyl ketone, methyliso-propyl ketone, methylpropyl ketone and methylisobutyl ketone are dissolved in polar ethers and esters such as diethylether, ethylacetate and the commercially commonly used solvents dimethylphthalate and 2,2,4-trimethylpentanediol-1,3-di-isobutyrate. These are also the solvents that are used in the above-mentioned WO 9932442, U.S. Pat. No. 3,326,809, and U.S. Pat. No. 3,151,170.

For safety reasons, Type 4 ketone peroxides can only be manufactured on a commercial scale in a solvent. This is not a problem for applications in which polar solvents can be used. However, when polar solvents are undesired, for instance in low-density polyethylene polymerization (LDPE), Type 4 ketone peroxides are not applicable for their low solubility in hydrocarbons. In such cases, Type 4 ketone peroxides cannot be used as a starting material. It is an object of the present invention to find Type 4 ketones that are sufficiently soluble in apolar hydrocarbon solvents and therefore can be used as starting materials in reactions where an apolar medium is a prerequisite.

It has now been surprisingly found that a safe composition of a ketone peroxide comprising

a) a peroxide derivative of the formula HOO—C(R₁)(R₂)—OOH

wherein

R₁ is a branched or unbranched alkyl group with 1 to 4 carbon atoms or alkenyl group with 2 to 4 carbon atoms; and

R₂ is a branched or unbranched alkyl or alkenyl group with 5 to 12 carbon atoms; and

b) a branched or unbranched hydrocarbon solvent;

the peroxide derivative of a) having a solubility of more than 40 g in 100 g of the solvent of b) at 20° C; and

comprises less than 10 wt. % of a peroxide derivative of the formula

HOO—C(R₁)(R₂)—OO—C(R₁)(R₂)—OOH,

wherein R₁ and R₂ have the previously given meanings.

The most suitable compositions thus obtained comprise ketone peroxide derivatives wherein both R₁ and R₂ represent alkyl groups, more particularly wherein one of these groups is a methyl group and the other an isoamyl or amyl group.

The requirement that the peroxide derivative has a solubility better than 40 g/100 g at 20° C., preferably better than 45 g/100 g, is fulfilled with common branched and unbranched hydrocarbon solvents, such as isododecane, dodecane, and isoparafins such as Isopar® E and Isopar® H. and commercially available solvents that are sold under the tradenames Norpar®, Shellsol®, and the like. Preferably, in the process according to the invention an apolar organic solvent is used having a dipole moment of less than 0.5D, most preferably 0D. Preferably, a saturated aliphatic hydrocarbon is used.

The solubility of the aforesaid Type 4 ketone peroxides in different solvents is given in Table I below, clearly demonstrating the unexpectedly high solubility of methyl-n-amyl ketone and methylisoamylketone derived Type 4 ketone peroxides in apolar hydrocarbon solvents.

In the first column, the ketone is given which is used for making the ketone peroxide derivative. In the second column R₁ and R₂ of the above formula are given. In columns 3 and 4, the solubility in g/100 g at 20° C. in polar dimethylphthalate and in apolar isododecane, respectively, are given.

TABLE I

| | | Solubility | |
|---|---|---|---|
| | | in | |
| | | dimethyl- | Solubility in |
| ketone | R₁/R₂ | phthalate | isodecane |
| methylethyl | R₁ = —CH₃ | >40 | <1 |
| ketone | R₂ = —CH₂CH₃ | | |
| methyl-n-propyl | R₁ = —CH₃ | >40 | 4 |
| ketone | R₂ = —CH₂CH₂CH₃ | | |
| methylisopropyl | R₁ = —CH₃ | >40 | 6 |
| ketone | R₂ = —CH(CH₃)₂ | | |
| methylisobutyl | R₁ = —CH₃ | >30 | 8 |
| ketone | R₂ = —CH₂CH(CH₃)₂ | | |
| methyl-n-amyl | R₁ = —CH₃ | >30 | >45 |
| ketone | R₂ = —CH₂(CH₂)₃CH₃ | | |
| methylisoamyl | R₁ = —CH₃ | >30 | >45 |
| ketone | R₂ = —CH₂CH₂CH(CH₃)₂ | | |

The peroxide compositions according to the invention may be advantageously used as a curing agent for unsaturated polyester and vinylester resins. The present compositions have the advantage over prior art compositions that they are free from phthalate and that they can be safely transported in IBCs (Intermediate Bulk Containers). Such resins usually include unsaturated polyester and one or more ethylenically unsaturated monomers. Suitable polymerizable monomers include styrene, α-methyl styrene, p-methyl styrene, chlorostyrenes, bromostyrenes, vinyl benzyl chloride, divinyl benzene, diallyl maleate, dibutyl fumarate, triallyl phosphate, triallyl cyanurate, diallyl phthalate, diallyl fumarate, methyl(meth)acrylate, n-butyl (meth)acrylate, ethyl acrylate, and mixtures thereof which are copolymerizable with the unsaturated polyesters. The unsaturated polyesters are, for example, polyesters as obtained by esterifying at least one ethylenically unsaturated di- or polycarboxylic acid, anhydride or acid halide, such as maleic acid, fumaric acid, glutaconic acid, itaconic acid, mesaconic acid, citraconic acid, allylmalonic acid, tetra-hydrophthalic acid, and others, with saturated and unsaturated di- or polyols, such as ethylene glycols, diethylene glycol, triethylene glycols, 1,2- and 1,3-propane diols, 1,2-, 1,3-, and 1,4-butane diols, 2,2-dimethyl-1,3-propane diols, 2-hydroxymethyl-2-methyl-1,3-propane diol, 2-buten-1,4-diol, 2-buten-1,4-diol, 2,4,4-trimethyl-1,3-pentane diol, glycerol, pentaerythritol, mannitol, and others. The di- or polycarboxylic acids may be partially replaced by saturated di- or polycarboxylic acids, such as adipic acid, succinic acid, and others and/or by aromatic di- or polycarboxylic acids, such as phthalic acid, trimellitic acid, pyromellitic acid, isophthalic acid, and terephthalic acid. The acids used may be tetrachlorophthalic acid and tetrabromophthalic acid.

The ketone peroxide compositions of the invention may be reacted with acid anhydride or acid halogen and halogen formate derivative in the manners that are common to the practitioner to give ketone peroxide derived bis-peroxyesters and bis-peroxycarbonates, respectively. A mixed ketone peroxide derived peroxyester-peroxycarbonate derivative is obtained when a mixture of acid anhydride or acid halogen and halogen formate is used. In this respect reference is made to WO 9932442.

The present invention therefore further relates to compositions of ketone peroxide derived bis-peroxyesters, bis-peroxycarbonates, or mixed ketone peroxide derived peroxyester-peroxycarbonates obtainable from the above-mentioned ketone peroxides, i.e. a composition of a ketone peroxide derived bis-peroxyester, bis-peroxycarbonate, or mixed peroxyester-peroxycarbonate comprising

a) a ketone peroxide derived bis-peroxyester, bis-peroxycarbonate, or mixed peroxyester-peroxycarbonate derivative of the formula

R₃[O]ₙC(O)OO—C(R₁)(R₂)—OOC(O)[O]ₙR₃

wherein

R₁ is a branched or unbranched alkyl group with 1 to 4 carbon atoms or alkenyl group with 2 to 4 carbon atoms; and

R₂ is a branched or unbranched alkyl or alkenyl group with 5 to 12 carbon atoms; and

R₃ is independently selected from a branched or unbranched alkyl group with 1 to 12 carbon atoms, alkenyl group with 2 to 12 carbon atoms; and an

aromatic group with 6-12 carbon atoms,

n is independently 0 or 1, and

b) a branched or unbranched hydrocarbon solvent; and

comprising less than 10 wt. % of a peroxide derivative of the formula

R₃[O]ₙC(O)OO—C(R₁)(R₂)—OO—C(R₁)(R₂)—OOC(O)[O]ₙR₃,

wherein R₁, R₂, R₃, and n have the previously given meanings.

Ketone peroxide derived monoperoxyesters and monoperoxycarbonates can be prepared by using per 1 mole of ketone peroxide 1 mole of the acid anhydride or acid halogen and halogen formate, respectively. In another aspect, the invention thus also pertains to a composition of a ketone peroxide derived monoperoxyester or monoperoxycarbonate comprising

a) a ketone peroxide derived monoperoxyester or monoperoxycarbonate derivative of the formula

HOO—C(R₁)(R₂)—OOC(O)[O]ₙR₃

wherein

R₁ is a branched or unbranched alkyl group with 1 to 4 carbon atoms or alkenyl group with 2 to 4 carbon atoms; and

R₂ is a branched or unbranched alkyl or alkenyl group with 5 to 12 carbon atoms; and

R₃ is selected from a branched or unbranched alkyl group'with 1 to 12 carbon atoms, alkenyl group with 2 to 12 carbon atoms; and an aromatic group with 6-12 carbon atoms;

n is 0 or 1, and

b) a branched or unbranched hydrocarbon solvent; and

comprising less than 10 wt. % of a peroxide derivative of the formula

HOO—C(R₁)(R₂)—OO—C(R₁)(R₂)—OOC(O)[O]ₙR₃,

wherein R₁, R₂, R₃, and n have the previously given meanings.

The ketone peroxides according to the present invention are prepared by a process wherein a ketone of the formula O═C(R₁)(R₂), wherein R₁ and R₂ have the previously given meanings, is reacted with hydrogen peroxide in a branched or unbranched hydrocarbon solvent in the presence of an acidic catalyst, in a manner that is known in the art. The acidic catalyst may be any common Lewis or Brönstedt acid. Usually mineral acids, such as diluted hydrochloric acid, are used to catalyze the reaction.

The peroxide compositions produced according to the preparation processes according to the present invention may be used as initiators for polymer production and in particular for the preparation of polyvinyl chloride, (meth)acrylic polymers, polystyrene, polyethylene, and copolymers comprising vinyl chloride, (meth)acrylates, styrene and/or ethylene, but they are equally suitable for curing unsaturated polyester or vinylester resins and for the modification of polymers (such as grafting of monomers onto the polymer, cross-linking, and/or degradation of the polymer).

The peroxide compositions of the present invention are suitable for grafting monomers onto polymers such as polyethers, polyolefins, and elastomers, and for the functionalization of polyolefins in the case of functional group-containing ketone peroxides of the present invention. In general, the peroxide may be brought into contact with the (co)polymer in various ways, depending upon the particular object of the modification process. For example, if surface modification of a three-dimensional polymeric object is desired, the ketone peroxide may be applied to the surface of the material to be modified. Alternatively, if it is desired to modify the (co)polymer homogeneously throughout the (co)polymeric matrix, then the peroxide may be mixed with the material to be modified, which material may be in the molten state, in the form of a solution, or, in the case of an elastomer, in a plastic state. It is also possible to mix the (co)polymer when in the powder or the granular form with the ketone peroxide.

The peroxide compositions are also suitable as an agent for the modification of polymers, such as polyethylene, polypropylene, polybutadiene, and copolymers of two or more olefins. Modification includes crosslinking, degradation, and grafting of monomers. Polymers may be in the liquid form, e.g., liquid rubbers. In general, any (co)polymer comprising abstractable hydrogen atoms, in particular polyolefins, can be modified by the present process. The (co)polymeric material treated by the process of the present invention may take any physical form including finely divided particles (flakes), pellets, film, sheet, in the melt, in solution, and the like. In the preferred embodiments of the present invention the (co)polymeric material is in the particulate form suitable for powder modification in a substantially oxygen-free atmosphere, in the melt form suitable for modification in an air-containing atmosphere or a nitrogen atmosphere, or in solution in a suitable solvent.

The amount of peroxide used in the modification process of the present invention should be an effective amount for achieving significant modification of the (co)polymer when treating a (co)polymer. More particularly, from 0.001-15.0% by weight of peroxide, based on the weight of the (co)polymer, should be employed. More preferably, from 0.005-10.0% by weight is employed. Most preferably, an amount of 0.01-5.0% by weight is employed. It is noted that the ketone peroxide may be pure T4 peroxide (as shown in the general formula) and comprises less than 10 wt. % of a peroxide derivative of the formula

HOO—C(R₁)(R₂)—OO—C(R₁)(R₂)—OOH,

wherein R₁ and R₂ have the previously given meanings. The presence of these small amounts of the corresponding T3 peroxide has no effect on its use as polymerization initiator, curing agent, and modifying agent.

The peroxide compositions can be prepared, transported, stored, and applied in the form of powders, granules, pellets, pastilles, flakes, slabs, pastes, solid master batches, and liquids. These formulations may have the form of dispersions, such as a suspension or an emulsion. The formulations may be phlegmatized if necessary, depending on the particular peroxide and its concentration in the formulation. Which of these forms is preferred depends partly on the application for which it will be used and partly on the manner in which it will be mixed. Also, considerations of safety may play a role to the extent that phlegmatizers may have to be incorporated into certain compositions to ensure their safe handling.

The formulations of the present invention are transportable, storage stable, and contain 1.0-90% by weight of one or more peroxides according to the present invention. Transportable means that the formulations of the present invention have passed the pressure vessel test (PVT). Storage stable means that the formulations of the present invention are both chemically and physically stable during a reasonable storage period under standard conditions.

More preferred formulations in accordance with the present invention contain 10-75% by weight of one or more of the ketone peroxides, most preferably these formulations contain 20-60% by weight of the ketone peroxides.

The formulations of the present invention can be liquids, solids, or pastes, depending on the melting point of the peroxide and the diluent employed.

Liquid formulations can be made using liquid phlegmatizers for the ketone peroxide, liquid plasticizers, organic peroxides, and mixtures thereof as the diluent. The liquid component generally is present in an amount of 1-99% by weight of the composition. Preferably, 10-90% by weight, more preferably 30-90% by weight, and most preferably 40-80% by weight of the liquid formulation consists of liquid diluents.

It should be noted that certain phlegmatizers may not be suitable for use with all of the ketone peroxides of the present invention. More particularly, in order to obtain a safe composition, the phlegmatizer should have a certain minimum flash point and a boiling point relative to the decomposition temperature of the ketone peroxide such that the phlegmatizer cannot be boiled off leaving a concentrated, unsafe ketone peroxide composition behind. Thus, the lower boiling phlegmatizers mentioned below may only be useful, for example, with particular substituted ketone peroxides of the present invention which have a low decomposition temperature.

The invention is further illustrated by the following examples:

### EXAMPLE 1

### Preparation of 2,2-dihydroperoxy-5-methylhexane (or 1,4-dimethylpentylidene dihydroperoxide or Type 4 methyl-isoamylketone hydroperoxide).

To a stirred mixture of 49.20 g of isododecane, 60.24 g of 5-methyl-2-hexanone, and 3.47 g of 5% aq. sulfuric acid were added 60.90 g of 70% aqueous hydrogen peroxide in 20 min keeping the temperature at 20° C. by cooling. The mixture was stirred for 30 min at 20° C. and the layers were allowed to separate. After removal of the water phase, 1.74 g of 5% aq. sulfuric acid were added at once and 30.44 g of 70% aq. hydrogen peroxide were added under stirring in 10 min, keeping the reaction temperature at 20° C. The mixture was stirred for 30 min at 20° C. and the layers were separated. The organic phase was washed twice with 39.18 g of 10% aq. sodium sulfate solution, yielding 146.4 g of product with 9.95% of active oxygen (% active oxygen for all examples is calculated as molar mass of active oxygen group(s) divided by the molar mass of peroxide). The product contained 45.87 wt. % of 1,4-dimethiylpentylidene dihydroperoxide, 4.84 wt. % of di(1-hydroperoxy-1,3-dimethylpentyl)peroxide, and 0.47 wt. % of hydrogen peroxide.

The product may be dried over magnesium sulfate or under vacuum. However, when the product is further reacted with acid halides, acid anhydrides, or chloroformates, drying is not required.

### EXAMPLE 2

### Preparation of 2,2-dihydroperoxyheptane (or 1-methylhexylidene dihydro-peroxide).

To a stirred mixture of 152.1 g of isododecane, 171.3 g of 2-heptanone and 10.0 g of 5% aq. sulfuric acid were added 174.9 g of 70% aq. hydrogen peroxide in 30 min keeping the temperature at 20-22° C. by cooling. The mixture was stirred for 30 min at 20° C. and the layers were allowed to separate. After removal of the water phase, 5.0 g of 5% aq. sulfuric acid were added at once and 87.5 g of 70% aq. hydrogen peroxide were added under stirring in 15 min, keeping the reaction temperature at 20° C. by cooling. The mixture was stirred for 30 min at 20° C. and the layers were separated. The organic phase was washed twice with 100 g of 10% aq. sodium sulfate solution, yielding 428.3 g of product containing 9.58% of active oxygen. The product contained 0.47 wt. % of hydrogen peroxide.

### EXAMPLE 3

### Preparation of 2-(isobutanoylperoxy)-2-(pivaloylperoxy)heptane in isododecane.

To 150 g of 1-methylhexylidene dihydroperoxide in isododecane, prepared as described in Example 2, were added under stirring 53.5 g pivaloyl chloride in 75 min keeping the temperature at 5-8° C. by cooling and maintaining the pH of the reaction mixture at 9.5 by simultaneous addition of 25% aq. sodium hydroxide. After the additions, the reaction mixture was stirred for 15 min at 10° C. and then 2.0 g 50% aq. benzyltrimethyl ammonium chloride were added, followed by addition of 56.7 g of isobutyrylchloride (99%) in 60 min, keeping the reaction temperature at 10° C. by cooling and maintaining the pH at 10.5 by simultaneous addition of 25% aq. sodium hydroxide. The mixture was stirred for another 30 min at 5° C. and pH 10.5. The layers were separated and the water phase was drained. Then 12.0 g of water and 3 g of tetrabutyl ammonium hydrosulfate were added to the organic phase followed by dosing of 30 g of isobutyrylchloride (99%) in 20 min under stirring, keeping the reaction temperature at 5° C. and maintaining the pH at 10.5 by addition of 25% aq. sodium hydroxide. The reaction mixture was stirred for another 30 min at 5° C. and pH 10.5 and the layers were separated. The organic phase was washed with water and 6% aq. sodium hydrogencarbonate and dried over MgSO₄.2H₂O yielding 184.3 g of desired product with an active oxygen content of 6.30%. Calculated assay based on active oxygen analysis: 63 wt. %.

### EXAMPLE 4

### Preparation of 1-hydroperoxy-1,4-dimethylpentyl-1-(peroxy-3,5,5-trimethyl-hexanoate).

To a stirred mixture of 126.8 g of isododecane, 144.2 g of methylisoamyl ketone, and 8.3 g of 5% aq. sulfuric acid were added under stirring 145.8 g of 7% aq. hydrogen peroxide in 20 min under cooling at 20° C. The mixture was stirred for 30 min at 20° C. and the layers were separated. After removal of the water phase, 4.15 g of 5% aq. sulfuric acid were added to the organic phase and under stirring 72.9 g of 70% aq. hydrogen peroxide were added under cooling at 20° C. Then the mixture was stirred for 30 min at 20° C. and the layers were allowed to separate. The organic phase was washed twice with 93.75 g of 10% aq. sodium sulfate and then 40 g of 20% aq. sodium chloride were added to the organic phase, followed by addition of 201.6 g of 3,5,5-trimethylhexanoyl chloride (98.6%) in 45 min keeping the reaction temperature at 10° C. by cooling and maintaining a pH of 9.5 in the reaction mixture by addition of 25% aq. sodium hydroxide. The mixture was stirred for another 30 min at 10° C. and then 50 g of water were added. The organic phase was washed once with 200 g of water and once with 200 g of 6% aq. sodium hydrogencarbonate yielding 471 g of desired product with an active oxygen content of 7.47%. Calculated assay: 71 wt. %.

### EXAMPLE 5

### Preparation of 2-(2-methylpropanoylperoxy)-2-(3,5,5-trimethylhexanoylperoxy)-5-methylhexane

To 100 g of 1,4-dimethylpentylidene dihydroperoxide in isododecane, prepared according to the preparation procedure of Example 1, were added under stirring 52.3 g of 3,5,5-trimethylhexanoyl chloride (98.6%) in 40 min keeping the temperature at 10° C. by cooling and maintaining the pH of the reaction mixture at 9.5 by simultaneous dosing of 20% aq. sodium hydroxide. The reaction mixture was stirred for another 15 min at 10° C. and then 4.0 g of 50% aq. benzyltrimethyl ammonium chloride and 4 g of potassium chloride were added, followed by addition of 41.3 g of isobutyrylchloride (99%) in 40 min, keeping the reaction temperature at 10° C. by cooling and maintaining the pH at 11.0 by simultaneous addition of 20% aq. sodium hydroxide. Water (12 g) was added to dissolve precipitated salts and so to improve pH reading.

Then, 2.0 g of 50% aq. benzyltrimethyl ammonium chloride were added, followed by addition of 10.0 g of isobutyrylchloride (99%) in 10 min under cooling at 10° C. and maintaining the pH at 11.0 by dosing of 20% aq. sodium hydroxide. The mixture was stirred for another 15 min at 10° C. and then 50 g of water were added. The organic phase was washed with water and 6% aq. sodium hydrogencarbonate and dried over MgSO₄.2H₂O yielding 122 g of desired product with active oxygen of 5.35%. Calculated assay based on active oxygen analysis: 63 wt. %.

### EXAMPLE 6

### Preparation of 2-(2-ethylhexyloxycarbonylperoxy)-2-isobutanoylperoxy-5-methylhexane

To 150 g of 1,4-dimethylpentylidene dihydroperoxide in isododecane, prepared as described above (Example 1) were added 3 g of 50% aq. benzyltrimethyl ammonium chloride and then under stirring 89.8 g of 2-ethylhexylchloroformate (99%) in 40 min keeping the temperature at 10° C. by cooling and maintaining the pH of the reaction mixture at 9.5 by simultaneous addition of 25% aq. sodium hydroxide. After the additions, the reaction mixture was stirred for 10 min at 10° C. and then 20 g of water were added, followed by addition of 58.3 g of isobutyrylchloride (99%) in 40 min, keeping the reaction temperature at 10° C. by cooling and maintaining the pH at 9.5 by simultaneous addition of 25% aq. sodium hydroxide. The mixture was stirred for another 20 min at 15° C. at pH 9.5 and 100 g of water were added. The layers were separated, the water phase was drained and the organic phase was dried over MgSO₄.2H₂O yielding 208.2 g of organic phase. To 190 g of this organic phase were added 2 g of tetrabutyl ammonium hydrosulfate and 20 g of water. Then, under stirring, 30 g of isobutyrylchloride (99%) were added in 20 min, keeping the reaction temperature at 20° C. and maintaining the pH at 9.5 by addition of 25% aq. sodium hydroxide. The reaction mixture was stirred for another 45 min at 20° C. at pH 9.5 and then 50 g of water were added. The layers were separated and the organic phase was washed twice with water and dried over MgSO₄.2H₂O yielding 182 g of desired product with active oxygen of 4.69%. Calculated assay based on active oxygen analysis: 57 wt. %.

### EXAMPLE 7

### Preparation of 2,2-di(acetylperoxy)-5-methylhexane

To a stirred mixture of 95.5 g Isopar H®, 117.0 g of methylisoamyl ketone and 6.74 g of 5% aq. sulfuric acid were added under stirring 118.2 g of 70% aq. hydrogen peroxide in 20 min under cooling at 20° C. The mixture was stirred for 30 min at 20° C. and the layers were separated. After removal of the water phase, 3.37 g of 5% aq. sulfuric acid were added to the reaction mixture and under stirring 59.1 g of 70% aq. hydrogen peroxide were dosed under cooling at 20° C. Then the mixture was stirred for 30 min at 20° C. and separated. The organic phase was washed twice with 76.2 g of 10% aq. sodium sulfate and then 141.0 g of acetic anhydride (97%) were added under stirring and cooling at 20° C. in 45 min keeping the pH at 7.0 by addition of 25% aq. sodium hydroxide. The reaction mixture was stirred for another 5 min at 20° C. and the layers were separated. After removal of the water phase 70.5 g of acetic anhydride.(97%) were added in 22.5 min under stirring and cooling at 20° C. During addition, the pH was maintained at pH 7.0 by addition of 25% aq. sodium hydroxide. The mixture was stirred for 5 min at 20° C. and the layers were separated. The organic phase was washed with 6% aq. sodium hydrogencarbonate and dried over MgSO₄.2H₂O yielding 279 g of desired product with active oxygen content of 7.30%. Calculated assay: 57 wt. %.

### EXAMPLE 8

In the following examples a laminate with a thickness of 4 mm was made at 20° C. A conventional Ortho UP resin (Ludopal P6®, ex BASF/DSM) in an amount of 100 pbw was combined with 0.5 pbw of a standard Co-octoate solution (Accelerator® NL49P, ex Akzo Nobel), 2 pbw of a ketone peroxide formulation, and glass fibre in such an amount that the total mixture contained 35% by weight of said glass fibre. A thermocouple was mounted in the middle of this laminate and time-temperature curves were measured. From the curve the following parameters were calculated:

Gel time (GT)=time in minutes elapsed between the starting point of the experiment (the moment at which the peroxide is mixed into the pre-accelerated UP resin) and 5.6° C. above the bath temperature (25.6° C.).

Time to peak exotherm (TTP)=time elapsed between the start of the experiment and the moment that the peak temperature is reached.

Peak Exotherm (PE)=the maximum temperature which is reached.

Barcol hardness (measured in the conventional way)=Time to reach barcol hardness 0-5 or 20-25 with Barcol meter 934. Accordingly, Barcol meter was placed onto the horizontal surface of laminate and pressed manually so that the indenter of the meter penetrates the surface and the hardness is directly read on the meter. Barcol meter number 934 was used for hard surfaces and number 935 is used for soft surfaces.

The residual styrene level was determined by conventional GC techniques after dissolving the sample in dichloromethane.

Curing of Unsaturated Polyesters.

Unsaturated polyester resins were cured at ambient temperature in accordance with the procedure given above. Methyl isoamyl ketonperoxide in isododecane (Example 1; MIAKP) was used. In comparative example A methyl ethyl ketonperoxide in dimethylphtalate (Butanox® M50, ex Akzo Nobel) was used. Both products have a total active oxygen content of 9% by weight and an active oxygen content H₂O₂ of 1% by weight. The results are summarized in the table below:

| | | | | | | Residual |
|---|---|---|---|---|---|---|
| | GT | TTP | | Barcol | Barcol | styrene |
| Example | (min) | (min) | PE (° C.) | 0-5 (h) | 20-25 (h) | (%) |
| 8 | 14.0 | 33.4 | 50 | 1.5 | 4 | 4.8 |
| A | 15.3 | 33.5 | 45 | 6 | >10 | 6.5 |

These results show that the MIAKP has a higher reactivity (higher PE), compared to standard Butanox® M50. Due to this higher peak exotherm a lower residual styrene content can be obtained. Furthermore, the MIAKP showed a higher efficiency in hardness built-up.

The new peroxide of Example 1 (MIAKP) has a higher reactivity due to its higher Type 4 content. This is an advantage over standard Butanox® M50. Moreover, this ketone peroxide can be produced in an apolar solvent, which means that the solution is phthalate free.

### EXAMPLE 9

Polymerization of Vinyl Chloride

The ketone peroxide bisester of Example 3 [2-(isobutanoylperoxy)-2-(pivaloylperoxy)heptane] of the present invention with a 1 hour half-life temperature at 49° C. was evaluated in vinyl chloride polymerization with good results. The polyvinyl chloride was produced according to an experimental procedure to be used for a 5 liter autoclave with the conversion being measured in time via the “butane tracer technique” (ref.: T. Y. Xie, A. E. Hamilec, P. E. Woods and H. Westmijze, J. Apl. Pol Sci, Vol 41(1990)). A 5 liter stainless steel reactor vessel was equipped with 1 baffle, a flat blade stirrer (n=700 rpm) a pressure transducer, a nitrogen purge, and a sampling device for the butane tracer technique, and was charged with 2200 g of demineralized water and 0.1% Alcotec® 72.5 on vinyl chloride and with 1 g of Na₂HPO₄ (ex Baker)+1 g of NaH₂PO₄ (ex Baker) as a buffer. The vessel was closed and pressurized with 15 bar nitrogen. The vessel was evacuated and pressurized with nitrogen (5 bar) for three times. Subsequently, the vessel was fed with 1100 ppm of 2-(isobutanoylperoxy)-2-(pivaloylperoxy)heptane vinyl chloride. The vessel was evacuated again and subsequently charged with 1100 g of vinyl chloride. The temperature was increased to the polymerization temperature of 57° C. The constant pressure time was 195 minutes. The standard polymerization time was 8 hours. Atmospheric pressure was attained before the vessel was opened and the vessel was evacuated for at least half an hour. The polyvinyl chloride formed was filtered and washed on a glass filter (S2). Subsequently, the polyvinyl chloride was dried in a fluid bed dryer at 60° C. and the yield of polyvinyl chloride amounted 90% on vinyl chloride monomers started.

1. A composition of a ketone peroxide comprising
a) a peroxide derivative of the formula
HOO—C(R₁)(R₂)—OOH
wherein
R₁ is a branched or unbranched alkyl group with 1 to 4 carbon atoms or alkenyl group with 2 to 4 carbon atoms; and
R₂ is a branched or unbranched alkyl or alkenyl group with 5 to 12 carbon atoms; and
b) a branched or unbranched hydrocarbon solvent;
the peroxide derivative of a) having a solubility more than 40 g in 100 g of the solvent of b) at 20° C.; and
comprises less than 10 wt. % of a peroxide derivative of the formula
HOO—C(R₁)(R₂)—OO—C(R₁)(R₂)—OOH,
wherein R₁ and R₂ have the previously given meanings.

2. The composition of claim 1 wherein R₁ and R₂ are alkyl groups.

3. The composition of claim 2 wherein R₁ is a methyl group and R₂ is an isoamyl or amyl group.

4. The composition of claim 1 wherein the solvent is a saturated aliphatic hydrocarbon.

5. A composition of a ketone peroxide derived bis-peroxyester, bis-peroxycarbonate, or mixed peroxyester-peroxycarbonate comprising
a) a ketone peroxide derived bis-peroxyester, bis-peroxycarbonate, or mixed peroxyester-peroxycarbonate derivative of the formula
R₃[O]ₙC(O)OO—C(R₁)(R₂)—OOC(O)[O]ₙR₃
wherein
R₁ is a branched or unbranched alkyl group with 1 to 4 carbon atoms or alkenyl group with 2 to 4 carbon atoms; and
R₂ is a branched or unbranched alkyl or alkenyl group with 5 to 12 carbon atoms; and
R₃ is independently selected from a branched or unbranched alkyl group with 1 to 12 carbon atoms, alkenyl group with 2 to 12 carbon atoms; and an aromatic group with 6-12 carbon atoms,
n is independently 0 or 1, and
b) a branched or unbranched hydrocarbon solvent; and
comprising less than 10 wt. % of a peroxide derivative of the formula
R₃ [O]ₙC(O)OO—C(R₁)(R₂)—OO—C(R₁)(R₂)—OOC(O)[O]ₙR₃,
wherein R₁, R₂, R₃, and n have the previously given meanings.

6. A composition of a ketone peroxide derived monoperoxyester or monoperoxycarbonate comprising
a) a ketone peroxide derived monoperoxyester or monoperoxycarbonate derivative of the formula
HOO—C(R₁)(R₂)—OOC(O)[O]ₙR₃
wherein
R₁ is a branched or unbranched alkyl group with 1 to 4 carbon atoms or alkenyl group with 2 to 4 carbon atoms; and
R₂ is a branched or unbranched alkyl or alkenyl group with 5 to 12 carbon atoms; and
R₃ is selected from a branched or unbranched alkyl group with 1 to 12 carbon atoms, alkenyl with 2 to 12 carbon atoms; and an aromatic group with 6-12 carbon atoms;
n is 0 or 1, and
b) a branched or unbranched hydrocarbon solvent; and
comprising less than 10 wt. % of a peroxide derivative of the formula
HOO—C(R₁)(R₂)—OO—C(R₁)(R₂)—OO C(O)[O]ₙR₃,
wherein R₁, R₂, R₃, and n have the previously given meanings.

7. A process for the preparation of a composition of claim 1 comprising the step wherein a ketone of the formula O═C(R₁)(R₂), wherein R₁ and R₂ have the previously given meanings, is reacted with hydrogen peroxide in the branched or unbranched hydrocarbon solvent in the presence of an acidic catalyst.

8. Use of the composition of claim 1 for polymerizing vinylchloride, (meth)acrylic monomers, styrene, ethylene, or mixtures thereof, for curing unsaturated polyester or vinylester resins, for grafting monomers onto a polymer, for crosslinking a polymer or for degrading a polymer.
